(19) United States
(12) Patent Application Publication
(10) Pub. No.: US 2005/0095221 A1
(43) Pub. Date: May 5, 2005

(54) Purified polyoxypropylene/polyoxyethylene copolymers and method of preparing the same

(76) Inventors: Mannarsamy Balasubramanian, Roswell, GA (US); R. Martin Emanuele, San Diego, CA (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/934,169

(22) Filed: Sep. 3, 2004

Related U.S. Application Data
(60) Provisional application No. 60/500,456, filed on Sep. 5, 2003.

(51) Int. Cl. A61K 31/785; C08G 59/14

(52) U.S. Cl. 424/78.36; 525/526

(57) ABSTRACT

The present invention comprises novel preparations of polyoxypropylene/polyoxyethylene octablock copolymers which retain the growth-promoting and immunity-enhancing activity of commercially-available preparations, but are free from the undesirable effects which are inherent in the prior art preparations. Because the polyoxypropylene/polyoxyethylene copolymers which comprise the present invention have a more homogenous population of molecules with fewer low molecular weight species than prior art preparations, the biological activity of the copolymer is better defined and more predictable. Moreover, the polyoxypropylene/polyoxyethylene copolymer of the present invention substantially reduces any risk to human health through the consumption of food animals since the copolymer hereof is not absorbed into the animal's edible tissue. Methods for preparation of the polyoxypropylene/polyoxyethylene copolymer of the present invention are also provided.

### CROSS-REFERENCE TO RELATED APPLICATIONS

This application claims priority from U.S. Provisional Patent Application No. 60/500,456, filed on Sep. 5, 2003, which document is hereby incorporated by reference to the extent permitted by law.

### STATEMENT REGARDING FEDERALLY SPONSORED RESEARCH & DEVELOPMENT

Not applicable.

### REFERENCE TO A SEQUENCE LISTING, TABLE, OR COMPUTER PROGRAM LISTING COMPACT DISK APPENDIX

Not applicable.

### BACKGROUND OF THE INVENTION

The present invention relates to purified polyoxypropylene/polyoxyethylene copolymers and a method for preparing and purifying polyoxypropylene/polyoxyethylene copolymers. In particular, the present invention relates to certain octablock polyoxypropylene/polyoxyethylene copolymers with reduced absorption characteristics in an animal's digestive tract wherein the copolymers contain a restricted amount of low molecular weight oligomeric impurities and a method of preparing and purifying polyoxypropylene/polyoxyethylene copolymers by restricting the amount of low molecular weight oligomeric species present in the copolymer preparation.

Improving the efficiency of food production and growth performance in food animals, such as beef cattle, poultry, and pigs, has long been a goal of the food animal industry. To meet this goal, food animal farmers have integrated nutritional supplements and low-level antibiotics into animal feed. The administration of small amounts of antibiotics such as tetracycline, penicillin, and sulfamethazine has been found to dramatically increase the growth performance of beef cattle, pigs and poultry. This for the reason that the efficiency of an animal's digestion is very dependent on the microorganisms that live naturally in its digestive tract. Some of these microorganisms improve digestion while others make it less effective. Added to the feed of pigs, poultry and cattle, low level antibiotics neutralize adverse microorganisms that live in the animal's digestive tract. Antibiotics help the intestine absorb more nutrients and water thereby helping the animal to grow well by making the best use of its food. The incorporation of antibiotics into animal feed also reduces the spread of infection from animal to animal. The vast majority of all beef cattle, swine, and poultry raised for human food consumption therefore consume antibiotics as part of their daily feed.

Despite the benefits attributable to the use of antibiotics in animal feed in terms of growth performance and feed efficiency, the extensive use of antibiotics has contributed to the emergence of antibiotic-resistant pathogens. In addition to being consumed by the food animal when mixed with the animal's feed, the antibiotics are also spread throughout the environment exposing external bacteria to the antibiotics. Constant exposure of both external and internal bacteria to antibiotics enables the bacteria to develop resistance to the antibiotics which can lead to a potentially uncontrollable bacteria present in the animal. Soil and water in the animal's environment may also be contaminated by antibiotic residues from animal waste. If the drug-resistant bacteria causes an infection in an animal or a human who has consumed the animal, the infection may not be controllable through treatment with conventional antibiotics. Moreover, a serious infection may decrease the time available to determine which antibiotic can be successfully used to treat the infection. Furthermore, the hazard of antibiotic-resistant bacteria to humans who consume these bacteria through meat is even more pronounced for those humans who are simultaneously being treated with an antibiotic. When humans take an antibiotic to treat the harmful bacteria that cause infection, many of the normal and beneficial bacteria present in the human body are also inhibited. This inhibition of normal bacteria may permit antibiotic-resistant bacteria to multiply quickly thereby causing an even more serious infection than that already being treated. The propagation of antibiotic-resistant pathogens in meat and the indiscriminate use of antibiotics to treat illness in humans thereby creating individual antibiotic resistance has resulted in a proliferation of pathogens that are rendering modern antibiotics useless.

As a result, the Food and Drug Administration's Center for Veterinary Medicine in conjunction with the U.S. Department of Agriculture and the Centers for Disease Control established The National Antimicrobial Resistance Monitoring System (NARMS) in 1996 to monitor changes in susceptibilities of human and animal enteric bacteria to several antibiotics. The NARMS program was expanded in 2001 and 2002 to include testing of retail meats and animal feed ingredients. In one study, researchers from the federal Centers for Disease Control and Prevention examined 407 samples of chicken from 26 supermarkets in four states: Georgia, Maryland, Minnesota and Oregon. The researchers found that 237 of the chicken samples were contaminated with the bacterium Enterococcus faecium, which was resistant to a potent combination of antibiotics. In another study, investigators from the U.S. Food and Drug Administration found that 20% of the 200 samples of ground turkey, chicken, beef and pork purchased at three Washington, D.C. supermarkets contained Salmonella. In addition, 84% of those bacteria were resistant to at least one type of antibiotic and 53% were resistant to at least three antibiotics. Nearly 1.4 million cases of Salmonella poisoning occur in the United States each year from eating contaminated beef, pork, poultry, eggs and milk. The risk is highest among elderly people and people whose immune systems do not function properly. The FDA is therefore considering a ban on the use of certain antibiotics. Many regulatory agencies worldwide have already banned, or are contemplating banning, the non-therapeutic use of all antibiotics in animals in an effort to prevent a continued threat to human health. Accordingly, there has developed a critical need in the food animal industry for non-antibiotic compounds that increase the efficiency of food production and growth performance in food animals.

Certain polyoxypropylene/polyoxyethylene copolymers have been found to have beneficial biological effects when administered to a human or animal. Of these, a group of polyoxypropylene/polyoxyethylene copolymers have been found to inhibit the growth of microorganisms, such as bacteria, yeast, and viruses. The biologic activity of these commercially-available copolymers are described in detail in U.S. Pat. Nos. 5,114,708 and 5,234,683 as having growth-stimulating and immunity-stimulating properties following administration to food animals. These compounds are composed of blocks of hydrophilic polyoxyethylene (POE) and hydrophobic polyoxypropylene (POP) built from a tetrafunctional initiator ethylenediamine. Typical commercially-available octablock copolymers have eight segments or blocks—four each of POP and POE. These copolymers have the general formula:
(H(C₃H₆O)_{b}(C₂H₄O)ₐ)₂NC₂H₄N((C₂H₄O)ₐ(C₃H₆O)_{b}H)₂
The mean aggregate molecular weight of the commercially-available octablock copolymer is between approximately 1500 and 40,000 daltons. “a” is a number such that the weight percentage of polyoxyethylene (C₂H₄O)ₐ blocks range between approximately 10% and 40% of the total molecular weight of the compound and have a molecular weight of between 176-1100 daltons. “b” is a number such that the weight percent of polyoxypropylene blocks of the total molecular weight range between approximately 60% and 90% of the compound and have a molecular weight of between 232-9900 daltons. While these compounds do not exhibit antibiotic or hormonal activity, they do possess growth performance-enhancing properties as well as immune-stimulating properties following administration to food animals in either feed or by injection.

It is believed that the biological actions of the synthetic octablock copolymer compounds that result in growth performance enhancement occur within the gastrointestinal (GI) tract of the target animal. While not wanting to be bound to the following theory, it is also believed that, since octablock copolymer compounds have both hydrophobic and hydrophilic domains, they act as surfactants and modulate partitioning coefficients between the contents of the GI tract and the epithelial cells which line the walls. By modulating this interaction, the block copolymers may contribute to increased absorption of poorly-absorbed dietary nutrients and limit adhesion and subsequent colonization of low-level enteric pathogens.

Because the biologic actions of synthetic octablock copolymers are believed to occur within the GI tract, the systemic absorption of these copolymers is neither necessary nor desirable. Absorption into the circulation would distribute the copolymers throughout the body where they could produce unwanted side effects in the target animal. Furthermore, a compound that distributes throughout the body of the target animal, raises human health safety issues because there may be residual levels of any absorbed compounds in the tissues of the animal that will ultimately be consumed by humans. For these reasons, a synthetic octablock copolymer that has reduced absorption through the GI tract following ingestion by a food animal would be desirable.

These synthetic polymers typically have a wide distribution of molecular chains and are characterized by their average molecular weights. Commercially-available polyoxypropylene/polyoxyethylene octablock copolymers typically have an average molecular weight of about 1500 daltons to about 40,000 daltons. Due to their relatively high average molecular weight, it is generally thought that these growth-promoting polymeric compounds will not be significantly absorbed following oral administration to a food animal.

However, because the commercially-available octablock copolymers typically contain significant levels of polymer chains with molecular weights of less than 4,000 daltons, the surprising discovery has been made that a small, but biologically active portion of the commercially-available octablock copolymers, may be absorbed into the tissue of the animal thereby presenting a hazard to humans when the food animal is consumed. A need in the art therefore exists for a compound that retains the same growth-enhancing effects of commercially-available octablock copolymers but contains a reduced amount of absorbable components thereby reducing the risk of absorption following oral administration. Accordingly, there is also a need in the art for a simple and relatively inexpensive method to selectively remove absorbable components present in octablock copolymers while still maintaining the growth-enhancing effects of the copolymers.

### BRIEF SUMMARY OF THE INVENTION

The present invention comprises novel preparations of polyoxypropylene/polyoxyethylene octablock copolymers which retain the growth-promoting and immunity-enhancing activity of commercially-available preparations, but are substantially free from the undesirable effects which are inherent in the prior art preparations. Because the polyoxypropylene/polyoxyethylene copolymers which comprise the present invention have a narrow molecular weight distribution and fewer low molecular weight species than prior art preparations, the biological activity of the copolymer is better defined and more predictable. Moreover, the polyoxypropylene/polyoxyethylene copolymer of the present invention substantially reduces any risk to human health through the consumption of food animals since the copolymer hereof is less subject to absorption into the animal's edible tissue.

The present invention comprises a polyoxypropylene/polyoxyethylene copolymer which has the following formula:
(H(C₃H₆O)_{b}(C₂H₄O)ₐ)₂NC₂H₄N((C₂H₄O)ₐ(C₃H₆O)_{b}H)₂
wherein the mean molecular weight of the copolymer is approximately 4000 to 10,000 daltons; “a” is a number such that the portion represented by POE constitutes approximately 5-20% by weight of the compound; and “b” is a number such that the POP portion of the total molecular weight of the block copolymer constitutes between approximately 80-95% by weight of the compound. The preferred copolymer preferably contains less than 4% by weight of low molecular weight components having a molecular weight of less than 4000 daltons.

A second embodiment of the polyoxypropylene/polyoxyethylene copolymer has the following formula:
(H(C₃H₆O)_{b}(C₂H₄O)ₐ)₂NC₂H₄N((C₂H₄₀)ₐ(C₃H₆O)_{b}H)₂
wherein the molecular weight of the composition is from about 4000 to 10,000 Daltons, “a” is a number such that the portion represented by polyoxyethylene constitutes from about 5-20% by weight of the composition, “b” is a number such that the portion represented by polyoxypropylene constitutes from about 80-95% by weight of the composition, and less than 50% by weight of the composition is absorbed through the gastrointestinal tract of the animal.

The present invention also includes methods for preparing polyoxypropylene/polyoxyethylene block copolymers with a narrow molecular weight distribution profile and fewer low molecular weight species than prior art preparations. The first method for preparing a purified polyoxypropylene/polyoxyethylene copolymer includes a solvent extraction technique wherein a polyoxypropylene/polyoxyethylene block copolymer composition is provided having the following formula:
(H(C₃H₆O)_{b}(C₂H₄O)ₐ)₂NC₂H₄N((C₂H₄O)ₐ(C₃H₆O)_{b}H)₂
wherein the mean molecular weight of the composition is from about 4000 to 10,000 daltons, “a” is a number such that the portion represented by polyoxyethylene constitutes from about 5-20% by weight of the composition, “b” is a number such that the portion represented by polyoxypropylene constitutes from about 80-95% by weight of the composition, and more than 4% by weight of the composition constitutes polymers having a molecular weight of less than 4000 daltons. The composition is mixed with water and a low-boiling, non-toxic solvent. The mixture is next separated so that at least two layers are formed wherein at least one of the layers contains a purified polyoxypropylene/polyoxyethylene block copolymer composition having less than 4 weight percent of polymers with a molecular weight of less than 4000 daltons. The purified composition is then extracted.

In a second method, a purified polyoxypropylene/polyoxyethylene block copolymer composition is prepared by first providing a polyoxypropylene/polyoxyethylene block copolymer composition having the following formula:
(H(C₃H₆O)_{b}(C₂H₄O)ₐ)₂NC₂H₄N((C₂H₄O)ₐ(C₃H₆O)_{b}H)₂
wherein the mean molecular weight of the composition is from about 4000 to 10,000 daltons, “a” is a number such that the portion represented by polyoxyethylene constitutes from about 5-20% by weight of the composition, “b” is a number such that the portion represented by polyoxypropylene constitutes from about 80-95% by weight of the composition, and more than 4% by weight of the composition constitutes polymers having a molecular weight of less than 4000 daltons. The composition is mixed with a low-boiling, non-toxic solvent and the resulting mixture is separated so that at least two layers are formed wherein at least one of the layers contains a purified polyoxypropylene/polyoxyethylene block copolymer composition having less than 4% by weight of polymers with a molecular weight of less than 4000 daltons. The purified composition can then be extracted for use.

In a third method, a purified polyoxypropylene/polyoxyethylene block copolymer composition is prepared by first providing a polyoxypropylene/polyoxyethylene block copolymer composition having the following formula:
(H(C₃H₆O)_{b}(C₂H₄O)ₐ)₂NC₂H₄N((C₂H₄O)ₐ(C₃H₆O)_{b}H)₂
wherein the mean molecular weight of the composition is from about 4000 to 10,000 daltons, “a” is a number such that the portion represented by polyoxyethylene constitutes from about 5-20% by weight of the composition, “b” is a number such that the portion represented by polyoxypropylene constitutes from about 80-95% by weight of the composition, and more than 4% by weight of the composition constitutes polymers having a molecular weight of less than 4000 daltons. A quantity of high pressure carbon dioxide is added to the composition and the mixture is stirred under pressure. The mixture is then separated to form at least two phases wherein the first phase contains the composition and the second phase contains a purified polyoxypropylene/polyoxyethylene block copolymer composition having less than 4% by weight of polymers with a molecular weight of less than 4000 daltons. The first phase is extracted thereby leaving the purified copolymer composition.

In a fourth method, a purified polyoxypropylene/polyoxyethylene block copolymer composition is synthesized de novo by first admixing respective quantities of an alkaline catalyst and a low molecular weight nitrogen compound wherein the compound is water-soluble. The mixture is then heated under vacuum. After reducing the temperature, a quantity of ethylene oxide is added to the mixture followed by the addition of a quantity of propylene oxide. The next step involves removing the catalyst by adding respective quantities of magnesium silicate, diatomaceous earth, and water. The mixture is then cooled and filtered through a pressure filter to produce a polyoxypropylene/polyoxyethylene block copolymer composition having the following formula:
(H(C₃H₆O)_{b}(C₂H₄O)ₐ)₂NC₂H₄N((C₂H₄O)ₐ(C₃H₆O)_{b}H)₂
wherein the mean molecular weight of the composition is from about 4000 to 10,000 daltons, “a” is a number such that the portion represented by polyoxyethylene constitutes from about 5-20% by weight of the composition, “b” is a number such that the portion represented by polyoxypropylene constitutes from about 80-95% by weight of the composition, and more than 4% by weight of the composition constitutes polymers having a molecular weight of less than 4000 daltons.

### BRIEF DESCRIPTION OF THE SEVERAL VIEWS OF THE DRAWING

FIG. 1 is a gel-permeation chromatograph of a commercially-available POP/POE copolymer;

FIG. 2 is a flow chart diagramming the extraction sequence of Example 3;

FIG. 3 is a flow chart diagramming the extraction sequence of Example 4;

FIG. 4 is a gel-permeation chromatograph of the hexane layer of Example 4;

FIG. 5 is a gel-permeation chromatograph of the water layer of Example 4;

FIG. 6 is a gel-permeation chromatograph of the top hexane layer of Example 5;

FIG. 7 is a gel-permeation chromatograph of the bottom hexane layer of Example 5;

FIG. 8 is a gel-permeation chromatograph of the top heptane layer of Example 5;

FIG. 9 is a gel-permeation chromatograph of the bottom heptane layer of Example 5;

FIG. 10 is a gel-permeation chromatograph of the top pentane layer of Example 5; and

FIG. 11 is a gel-permeation chromatograph of the bottom pentane layer of Example 5.

### DETAILED DESCRIPTION OF THE INVENTION

As illustrated by Examples 5-7 below, Applicants have discovered that the relative size of an octablock copolymer molecule is a significant factor in determining the probability for its absorption. The lower molecular weight components of the octablock copolymers are more likely to be absorbed into the gastrointestinal system. As used herein, low molecular weight components are those having a molecular weight of less than 4000, less than 3500, less than 3000, less than 2500, less than 2000, less than 1750, less than 1500, less than 1250, and less than 1000 daltons.

Thus, the present invention is directed to purified octablock copolymers with reduced low molecular weight components that may be absorbed into the tissues of food animals. The octablock copolymers of the present invention may be prepared by removing the undesirable molecules from commercially-available octablock copolymers or by synthesizing the octablock copolymer de novo with fewer absorbable components than are normally present in commercially available octablock copolymers.

The preferred composition of the present invention comprises a surface active copolymer. The surface active copolymer can be an ethylene oxide-propylene oxide condensation product with the following formula:
(H(C₃H₆O)_{b}(C₂H₄O)ₐ)₂NC₂H₄N((C₂H₄O)ₐ(C₃H₆O)_{b}H)₂
wherein the mean molecular weight of the copolymer is from about 4,000 to 10,000 daltons, more preferably from about 6000 to 9000 daltons, and most preferably from about 7000 to 8000 daltons. “a” is a number such that the portion represented by polyoxyethylene constitutes from about 5-20% by weight of the compound, more preferably from about 7-17%, and most preferably from about 9-15%. “b” is a number such that the polyoxypropylene portion of the total molecular weight of the octablock copolymer constitutes approximately 80-95% by weight of the copolymer, more preferably 83-92%, and most preferably 85-91%. The preferred copolymer preferably contains less than 4% by weight, more preferably less than 2%, and most preferably less than 1% of oligomeric impurities having a molecular weight of less than 4000 daltons, more preferably less than 3000 daltons, and most preferably less than 2000 daltons. In another preferred embodiment of the copolymer, less than 50% by weight, preferably less than 40%, more preferably less than 30%, more preferably less than 20%, and most preferably less than 10% of the composition is absorbed through the gastrointestinal tract of the animal.

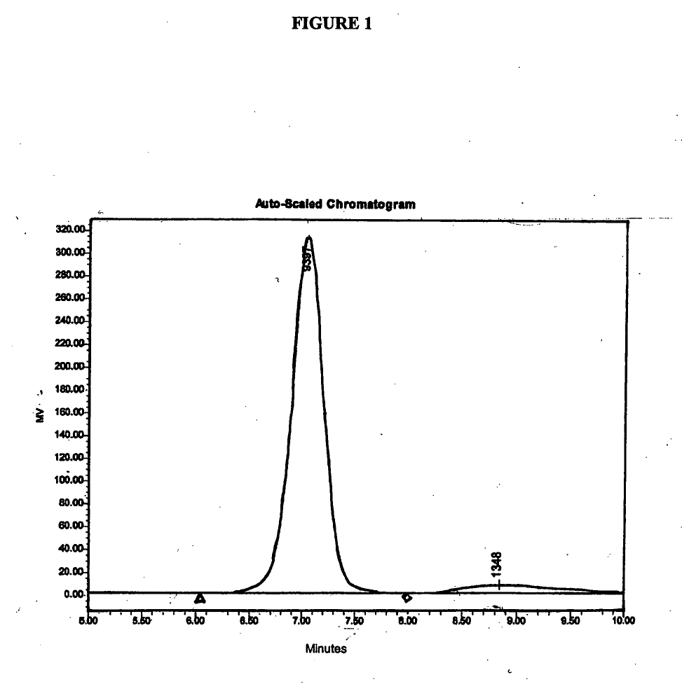

The purified polyoxypropylene/polyoxyethylene copolymer of the present invention can be prepared using solvent extraction techniques according to the methods of the present invention wherein low molecular weight oligomers are substantially removed from a commercially-available octablock copolymer such as CRL-8761 manufactured by BASF corporation. As can be seen in the gel permeation chromatograph shown in FIG. 1., commercial grade CRL-8761 is composed of a broad distribution of molecules with a peak molecular weight of approximately 9000 to 9500 daltons. FIG. 1 also shows a small secondary peaks or shoulders at the low molecular weight side of the primary peak. This area of the CRL-8761 chromatogram represents the low molecular weight molecules present in the sample. The peak molecular weight of low molecular weight species range in size from approximately 1250 to 1350 daltons. It is believed that these low molecular weight oligomers are more easily absorbed into the tissue of a target animal following consumption of feed treated with the prior art copolymer composition. Using the method of the present invention, most of these low molecular weight species are extracted from the copolymer thereby leaving primarily high molecular weight species in the composition which are too large to pass from the gastrointestinal system of a target animal into that animal's edible tissue.

A first method of the present invention comprises a solvent extraction technique involving the preparation of a polyoxypropylene/polyoxyethylene octablock copolymer and solvent mixture to which is then added water. A single solvent or multiple solvents may be used. The preferred solvents are non-toxic and low-boiling and include, but are not limited to, high pressure or liquid carbon dioxide, acetone, alcohols including methanol and ethanol, and hydrocarbon solvents including propane, butane, pentane, hexane, and heptane with hexane being the most preferred. In practicing the present invention, the copolymer/solvent/water mixture is separated into at least three layers: a top solvent layer, a middle water layer and a bottom water layer. According to the present invention, the top solvent layer generally contains a small percentage of the copolymer having a substantially high percentage of low molecular weight species therein. The middle and bottom water layers generally contain a large percentage of the copolymer having a substantially low percentage of low molecular weight species. Both the solvent layer containing the low molecular weight species and the water layers containing the purified copolymer may be washed and extracted several times to further remove low molecular weight species from the starting material.

In a second method, the copolymer is not mixed with water, but is mixed directly with at least one solvent. The mixture is then separated thereby obtaining at least two layers wherein the low molecular weight species of the copolymer are present in the top solvent layer and the purified copolymer can then be extracted from the bottom layer. The bottom layer may, if desired, be washed and extracted several times to further remove low molecular weight species from the starting material.

In a third method, a solvent wash using high pressure carbon dioxide or liquid carbon dioxide is used to purify samples of a commercially-available octablock copolymer. In this method, the copolymer is loaded into a high-pressure stainless steel vessel equipped with a stirrer. The copolymer can be used alone or with an absorptive material such as diatomaceous earth. While stirring the contents in the reactor, compressed fluid CO₂ is pumped into the reactor over a period of time. The dissolved or extracted components of the copolymer are then isolated from the solvent stream by lowering the CO₂ pressure sufficiently to cause phase separation. The separated copolymer having a substantially large percentage by weight of low molecular weight species is isolated and removed. Recovered CO₂ is fed back to the solvent circulation loop. Extraction may then be continued for a period of time with additional CO₂ fluid pumped into the reactor under increased pressure. Once again, separated copolymer having a substantially large percentage by weight of low molecular weight components is removed and isolated. The extraction is again continued under similar conditions and additional copolymer with a significant percentage of low molecular weight components is then removed and isolated. After extraction is complete, the substantially-pure copolymer having less than 4% by weight of low molecular weight components can then be removed from the vessel. It will be appreciated by one skilled in the art that high pressure or liquid CO₂ can also be mixed with a co-solvent, such as methanol, and this solvent mixture used in the extraction method described above.

In a fourth method, a polyoxypropylene/polyoxyethylene octablock copolymer is synthesized de novo by the addition of an alkaline catalyst, such as potassium hydroxide or cesium hydroxide, to a low molecular weight water-soluble nitrogen compound, such as ethylenediamine. The mixture is heated under vacuum then cooled. The next step includes the sequential addition of ethylene oxide followed by propylene oxide to which is then added magnesium silicate, diatomaceous earth, and water. The complete mixture is then cooled and filtered through a pressure filter thereby producing the purified octablock copolymer of the present invention. If additional removal of low molecular weight species from the purified octablock copolymer is desired, it is contemplated that the solvent extraction methods described above could also be used on the final product from the copolymer synthesis method.

It should be understood that, in the following examples and in accordance with the present invention, molecular weight is preferably measured by gel permeation chromatography (GPC). The GPC unit is calibrated using a polymer of known molecular weight and of chemical similarity to the compound being tested. In the present invention, polyethylene oxide (100% PEO) standards were used to calibrate the GPC unit, and molecular weight was calculated using PEO standards which is the same as polyethylene glycol (100% PEG) standards. In accordance with the present invention, molecular weight is therefore measured using the following GPC equipment: HPLC equipment, Waters 510 pump, 717 Plus autosampler, HR3 Waters Styragel columns and Waters 410 RI detector at 35° C. with a mobile phase of THF at 1.0 ml per minute. Samples are prepared by making a solution of 0.2% by weight of the copolymer in THF solvent and injected into the GPC system. The peak molecular weights of main peak and low molecular weight peak are calculated using PEG standards. One skilled in the art will appreciate that the molecular weight numbers calculated using PEO or PEG standards might be slightly different than actual molecular weight if measured using absolute methods such as light scattering or MALDI mass spectrometry. One skilled in the art will also appreciate that, to enhance accuracy, it is important to generate data from several batches of copolymer tested over a period of time. Finally, it should be understood that variations in molecular weight measurements under similar test conditions will occur and such variations can generally be attributed to batch to batch variation in manufacturing and day to day variation in analytical tests.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

The chemical profile of a commercially-available octablock copolymer sold under the brand name CRL-8761 by BASF Corporation was determined by first mixing the copolymer in its steel drum shipping container. This was accomplished by rolling the drum horizontally over a roller mixer for approximately two hours at 30 rpm. A small portion of the homogenous copolymer was then siphoned and transferred to a glass bottle. Two samples were taken from the bottle and profiled using the following high performance liquid chromatography (HPLC) equipment: Waters 510, 717 Plus autosampler, 500 A and 1000 A Styragel columns, and a 410 RI detector. Tetrahydrofuran (THF) was used as a mobile phase at 1.0 ml per minute. The two samples were each injected twice and the injections for each sample were repeated the following day. The peak molecular weights of the main peaks and low molecular weight peaks of both samples were calculated using PEG standards. The average peak molecular weight of the main peak of both samples was 9283 daltons. The average peak molecular weight of the low molecular weight peaks of both samples was 1323 daltons. Thus, the low molecular weight species constituted 5.13% by weight of the commercially-available CRL-8761 compound.

In the typical gel-permeation chromatograph for CRL-8761 shown in FIG. 1, a main peak is shown with an approximate retention time of between 6.5 and 7.5 minutes and a much smaller secondary peak is shown with retention times of between 8.5 and 9.5 minutes. In addition, there are oligomeric materials at a retention time of 10-10.5 minutes, however, these are poorly resolved in FIG. 1. The retention times correspond to average molecular weights of about 9,000 daltons for the main peak and about 1,500 daltons for the secondary peak.

### Example 2

Using the commercially-available CRL-8761 profiled above, a study was conducted to determine the relative permeability or absorption rates of the low molecular weight components and high molecular weight components of the poloxamer. In this study, the cell culture experiments were performed with Caco2 cell lines which are commonly used to study passive drug absorption. Specifically, the Caco2 cell lines were used to identify how and at what rate the CRL-8761 was transported through the intestinal epithelium. After an initial equilibration period, Caco2 cells in the range of 6-7×10⁴ per well were seeded onto polyester membrane cell culture inserts, namely, Costar Transwell-Clear 3470 having a 0.4 μm pore size and a growth area of 0.33 cm². The inner and outer chambers of the inserts were filled with 0.4 mL and 0.5 mL, respectively, of cell culture medium (Vitacell Minimum Essential Medium alpha-Sigma Corporation-M0894-3H172) with 10% of fetal bovine serum and 1% of antibiotic antimycotic solution. The plates with inserts were then incubated at 37° C. with 95% air and 5% CO₂. Trans Epithelial Electrical Resistance (TEER) measurements were made periodically to follow the monolayer growth using MILLICELL-ERS. After approximately 21 days, the plates were then used for the permeability study.

### Example 3

Utilizing a homogenizer, CRL-8761 was directly dissolved in cell culture medium at a concentration of about 10% by weight. A TEER measurement was made before the addition of the CRL-8761-infused medium to the cell layers of the plates discussed above. The CRL-8761-infused medium was placed inside the inner chamber and a quantity of neat cell culture medium was added to the outer chamber. The cells were then allowed to incubate. After a predetermined time interval, the samples from the inner chamber and outer chamber were collected and placed into separate test tubes and then vacuum dried.

### Example 4

Samples for GPC analysis were prepared by adding 0.3 ml to 1 ml of THF into the test tubes containing the dried inner and outer chamber samples. The test tubes were then vortexed or at least one minute at room temperature. The mixtures were then filtered through a Gelman Acrodisc 0.2 micron nylon filter. 150 μL of each test sample was injected into the GPC using an autosampler. The results of the GPC analysis are shown below in Table 1.

TABLE 1
GPC Molecular Weight Characteristics of Inner
and Outer Chamber Samples

| Sample ID- | Main peak, | area % for | Low Mwt peak | area % for low |
|---|---|---|---|---|
| chamber | area, mV | Main peak | area, mV | Mwt peak |
| I81-inner | 93489 | 97.51 | 2391 | 2.49 |
| O80-outer | 1115 | 88.70 | 142 | 11.3 |
| 2I48-inner | 14383 | 97.94 | 302 | 2.06 |
| 2O48-outer | 6096 | 95.03 | 319 | 4.97 |
| 3I24-inner | 173 | 33.02 | 351 | 66.98 |
| 6O48-outer | 97 | 78.23 | 27 | 21.77 |
| 5O24-outer | 255 | 60.71 | 165 | 39.29 |
| 4O8-outer | 10 | 58.82 | 7 | 41.18 |
| 6I48-inner | 172930 | 52.16 | 158631 | 47.84 |
| 5I24-inner | 9040 | 94.96 | 480 | 5.094 |
| 4I8-inner | 41433 | 96.74 | 1395 | 3.26 |
| 6O48-outer | 9 | 11.84 | 67 | 88.16 |
| 5O24-outer | 104 | 39.69 | 158 | 60.31 |
| 4O8-outer | 19 | 86.36 | 3 | 13.64 |

### Example 5

In a second set of studies, rather than using the same insert for all three time points, the entire contents of the insert was used for each time point. For example, at 8 hours, the entire contents from an insert inner chamber was removed and dried to provide a sample for GPC analysis. Other inserts were continued for 24 and 48 hours and samples taken at these time points, respectively. 150 μL samples were injected and analyzed by GPC. The peaks corresponding to the main high molecular weight components and the low molecular weight components were measured and compared for the different samples taken. The GPC analysis results are shown in Table 2.

TABLE 2
GPC Molecular Weight Characteristics of Full Insert Samples

| | | | Low | |
|---|---|---|---|---|
| Sample ID- | Main peak, | area % for | Mwt peak, | area % for low |
| chamber | area, mV | main peak | area, mV | Mwt. peak |
| I18-inner | 3228 | 81.76 | 720 | 18.24 |
| I18-inner | 2733 | 80.74 | 652 | 19.26 |
| O18-outer | | 0.00 | 6 | 100.00 |
| O18-outer | | 0.00 | 3 | 100.00 |
| I124-inner | 603 | 91.52 | 55.9 | 8.48 |
| I124-inner | 620 | 43.60 | 802 | 56.40 |
| O124-outer | | 0.00 | 22 | 100.00 |
| O124-outer | | 0.00 | 18 | 100.00 |
| I148-inner | 9.8 | 1.92 | 501 | 98.08 |
| I148-inner | 6.8 | 2.03 | 328 | 97.97 |
| I28-inner | 1780 | 64.54 | 1022 | 36.47 |
| I28-inner | 1236 | 65.71 | 645 | 34.29 |
| O28-outer | | 0.00 | 6.3 | 100.00 |
| O28-outer | | 0.00 | 1.7 | 100.00 |
| I224-inner | 790 | 52.25 | 722 | 47.75 |
| I224-inner | 666 | 53.11 | 588 | 46.89 |
| O224-outer | | 0.00 | 23 | 100.00 |
| O224-outer | | 0.00 | 22 | 100.00 |
| I248-inner | 37 | 6.31 | 549 | 93.69 |
| I248-inner | 24 | 5.00 | 456 | 95.00 |
| O248-outer | 7 | 2.12 | 323 | 97.88 |
| O248-outer | 7 | 2.20 | 311 | 97.80 |
| I38-inner | 768 | 35.87 | 1373 | 56.02 |
| I38-inner | 598 | 26.60 | 1650 | 73.40 |
| O38-outer | | 0.00 | 10 | 100.00 |
| O38-outer | | 0.00 | 5 | 100.00 |
| I324-inner | 1062 | 43.98 | 1353 | 56.02 |
| I324-inner | 893 | 37.18 | 1509 | 62.82 |
| O324-outer | | 0.00 | 8.44 | 100.00 |
| O324-outer | | 0.00 | 8.383 | 100.00 |

As shown above, the area under the curve for the second peak corresponding to the low molecular weight components is consistently higher for the outer chamber samples than the inner chamber samples. Almost all of the GPC curves for the outer chamber samples had peaks for only low molecular weight components thereby demonstrating that the low molecular weight components preferably transported through the Caco2 cell layer than the main high molecular weight components.

### Example 6

Under the same conditions as Example 5, a third series of samples were analyzed by GPC. The results are shown below in Table 3.

TABLE 3
GPC Molecular Weight Characteristics of Full Insert Samples

| | | | Low | |
|---|---|---|---|---|
| Sample ID- | Main peak, | area % for | Mwt peak, | area % for low |
| chamber | area, mV | main peak | area, mV | Mwt. peak |
| I18-inner | 3898 | 96.06 | 160 | 3.94 |
| I18-inner | 3652 | 96.23 | 143 | 3.77 |
| O18-outer | 16 | 69.57 | 7 | 30.43 |
| O18-outer | 17 | 65.38 | 9 | 34.62 |
| I124-inner | 1900 | 90.61 | 197 | 9.39 |
| I124-inner | 1409 | 90.38 | 150 | 9.62 |
| O124-outer | 0 | 0.00 | 6.8 | 100.00 |
| O124-outer | 0 | 0.00 | 4.6 | 100.00 |
| I148-inner | 10277 | 98.78 | 127 | 1.22 |
| I148-inner | 10506 | 98.82 | 125 | 1.18 |
| O148-outer | 8.275 | 80.54 | 2 | 19.46 |
| O148-outer | 33 | 84.62 | 6 | 15.38 |
| I28-inner | 5154 | 96.68 | 177 | 3.32 |
| I28-inner | 1020 | 82.52 | 216 | 17.48 |
| O28-outer | | 0.00 | 18 | 100.00 |
| O28-outer | | 0.00 | 16 | 100.00 |
| I224-inner | 706 | 76.66 | 215 | 23.34 |
| I224-inner | 490 | 77.53 | 142 | 22.47 |
| O224-outer | | 0.00 | 15 | 100.00 |
| O224-outer | | 0.00 | 12 | 100.00 |
| I248-inner | 208 | 69.10 | 93 | 30.90 |
| I248-inner | 168 | 70.59 | 70 | 29.41 |
| O248-outer | 14 | 43.75 | 18 | 56.25 |
| O248-outer | 259 | 95.22 | 13 | 4.78 |
| I38-inner | 9268 | 98.75 | 117 | 1.25 |
| I38-inner | 8529 | 98.77 | 106 | 1.23 |
| O38-outer | 23 | 33.82 | 45 | 66.18 |
| O38-outer | 30 | 48.39 | 32 | 51.61 |
| I324-inner | 10056 | 98.72 | 130 | 1.28 |
| I324-inner | 7826 | 98.58 | 113 | 1.42 |
| O324-outer | 24 | 36.36 | 42 | 63.64 |
| O324-outer | 15 | 31.91 | 32 | 68.09 |
| I348-inner | 14774 | 98.89 | 166 | 1.11 |
| I348-inner | 7718 | 98.91 | 85 | 1.09 |
| O348-outer | 20 | 32.26 | 42 | 67.74 |
| O348-outer | 21 | 35.00 | 39 | 65.00 |

As shown above, the trend observed in Example 5 is reproduced in Example 6. The area under the curve for the second peak corresponding to the low molecular weight components of the poloxamer is consistently higher for the outer chamber samples than for the inner chamber samples thereby demonstrated that the low molecular weight components are preferentially transported through the Caco2 cell layer over the main high molecular weight components.

### Example 7

While some of the anomalies observed in the data generated in Examples 5 and 6 could be explained as typical biological experimental errors, an investigation was conducted in order to identify possible causes for certain inner chambers retaining a significant amount of high molecular weight components. In this investigation, it was discovered that some of the inserts appeared to have developed holes in the monolayer during the permeability studies thereby indicating that the monolayers may not be viable at longer sampling intervals. TEER measurements were therefore taken in order to study the viability and integrity of the monolayers. TEER values for the control inserts were measured in the presence of media with no CRL-8761 copolymer present. TEER values for sample inserts were measured either in the presence of CRL-8761 copolymer/media mixture (during the experiment) or in the presence of media (after removing the contents of the insert for sampling, e.g., 24 hour TEER value for 8 hour sampling inserts). The TEER measurements are shown below in Table 4.

TABLE 4
TEER Measurement Results.

| Insert # | 0 Hours | 8 Hours | 24 Hours | 48 Hours |
|---|---|---|---|---|
| Control 1 | 1310 | 1552 | 1328 | |
| Control 2 | 1450 | 1570 | 1385 | |
| Control 3 | 1152 | 1195 | 1190 | |
| Insert 8-1 | 1783 | 1325 | 1930 | 1643 |
| Insert 8-2 | 1370 | 1094 | 1056 | 1007 |
| Insert 8-3 | 1485 | 1245 | 1520 | 1370 |
| Insert 8-4 | 1350 | 1080 | 1149 | 1150 |
| Insert 24-1 | 1545 | 1286 | 505 | 984 |
| Insert 24-2 | 1350 | 1176 | 480 | 750 |
| Insert 24-3 | 1557 | 1256 | 475 | 798 |
| Insert 24-4 | 1600 | 1146 | 466 | 745 |

The results of this study show that the TEER values did not change with the control samples. In addition, removing the CRL-8761 copolymer/media mixture from the inserts after 8 hours of study did not affect the TEER values when measured after 24 hours and 48 hours. The TEER measurements therefore demonstrate that, under normal cell growth conditions, the integrity of the cell monolayers was compromised after keeping the cells in contact with the copolymer for more than 8 hours. Thus, when interpreting the permeability results, it is the 8-hour samples that should be considered. This is an acceptable condition because standard permeability studies are conducted for 8 hours.

### Example 8

Under the same conditions as Example 5, a fourth series of samples were analyzed by GPC to confirm the trends observed in previous examples. The results are shown below in Table 5.

TABLE 5
GPC Molecular Weight Characteristics of Full Insert Samples

| | | | | area |
|---|---|---|---|---|
| Sample ID- | Main peak, | area % for | Low Mwt peak, | % for low |
| chamber | area, mV | main peak | area, mV | Mwt. peak |
| I18-inner | 60629 | 94.64 | 3433 | 5.36 |
| O18-outer | 23.27 | 59.90 | 15.58 | 40.10 |
| I28-inner | 8391 | 90.00 | 932 | 10.00 |
| O28-outer | 0 | 0.00 | 12.51 | 100.00 |
| I38-inner | 2377 | 90.07 | 262 | 9.93 |
| O38-outer | 0 | 0.00 | 20.68 | 100.00 |
| I48-inner | 4773 | 85.05 | 839 | 14.95 |
| O48-outer | 9.549 | 32.08 | 20.216 | 67.92 |
| I224-inner | 12816 | 92.83 | 990 | 7.17 |
| I324-inner | 10010 | 92.19 | 848 | 7.81 |
| I424-inner | 11018 | 92.22 | 930 | 7.78 |

As shown above, the permeability trend observed in previous examples were reproduced in this example as well particularly in the 8 hour samples. The area under the curve for the second peak, corresponding to the low molecular weight components in CRL-8761, was consistently higher for the outer chamber samples than the inner chamber samples. This demonstrates that the low molecular weight components preferably transported through the Caco2 cell layer over the high molecular weight components.

### Example 9

To purify the polymers discussed above, one gram of commercial grade CRL-8761 was added to approximately 5 grams of hexane and mixed to form a clear solution. 1 gram of distilled water was then added to the copolymer/hexane mixture and mixed thoroughly. This new mixture was centrifuged for 40 minutes and three layers were obtained: a clear top layer, a cloudy middle layer and a cloudy bottom layer. The top layer (385-52-1) and middle layer (385-52-2) were sampled, dried and analyzed by GPC. The results are shown in Table 6.

TABLE 6
GPC Molecular Weight Characteristics of
CRL-8761 after Hexane Washing

| | Sample | Peak Mwt. of | Peak Mwt. of | Percent |
|---|---|---|---|---|
| Sample | ID | Main Peak | Low Mwt. Peak | low Mwt. |
| CRL-8761 | 385-50-1 | 8797 | 1161 | 7.24 |
| CRL-8761 | 385-50-1 | 8816 | 1173 | 6.93 |
| Hexane | 385-52-1 | 8609 | 1229 | 27.49 |
| Top Layer | | | | |
| Middle | | 8870 | | 2.93 |
| Layer | | | | |

The results of this analysis indicate that extraction of water solution of CRL-8761 concentrates the low molecular weight components in the hexane layer while purified polymer is left in the water layer.

### Example 10

7.2 g of CRL-8761 was mixed with 7.6 g of water and 22.0 g of hexane. The mixture was centrifuged and four layers were obtained: a clear top hexane layer, a middle clear layer, a middle cloudy layer, and a bottom viscous white layer. The bottom three layers were sampled and dried to estimate the solid content in each layer.

The hexane top layer was again extracted with 2.4 g of water. It was then centrifuged and two layers were obtained. The top and bottom layers were sampled and dried to estimate the solid content.

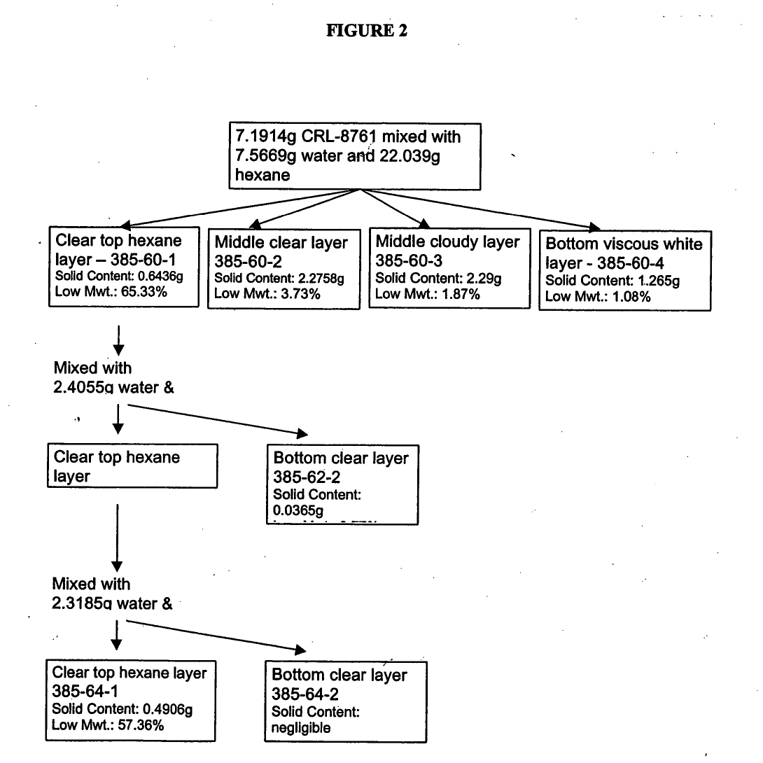

The hexane top layer from the second extraction wash was again extracted with 2 g of water and centrifuged. Two layers were again obtained and sampled and dried to estimate the solid content in each layer. This extraction sequence is shown as a flow chart diagram in FIG. 2. The results of the hexane extraction is shown in Table 7.

TABLE 7
Partition of CRL-8761 in first stage hexane extraction

| Starting | Hexane Layer from 1^{st} | Water Layers from 1^{st} |
|---|---|---|
| Material | Extraction | Extraction |
| CRL-8761 | 9% of starting | 81% of starting |
| with 6.74% | material with 65% | material with |
| low Mwt. | low Mwt. | 1.1-3.7% low |
| content | component | Mwt. component |

*The solid contents in these fractions do not equal 100% due to experimental error and handling loss.

### Example 11

7.3305 g of CRL-8761 were mixed and dissolved in 22.1 g of hexane to form a clear solution. To the hexane solution was added 7.0 g of water which was then mixed thoroughly. The mixture was centrifuged and three layers were obtained: a clear top hexane layer, a middle clear layer, and a bottom viscous white layer. The bottom two layers were sampled and dried to estimate the solid content in each layer.

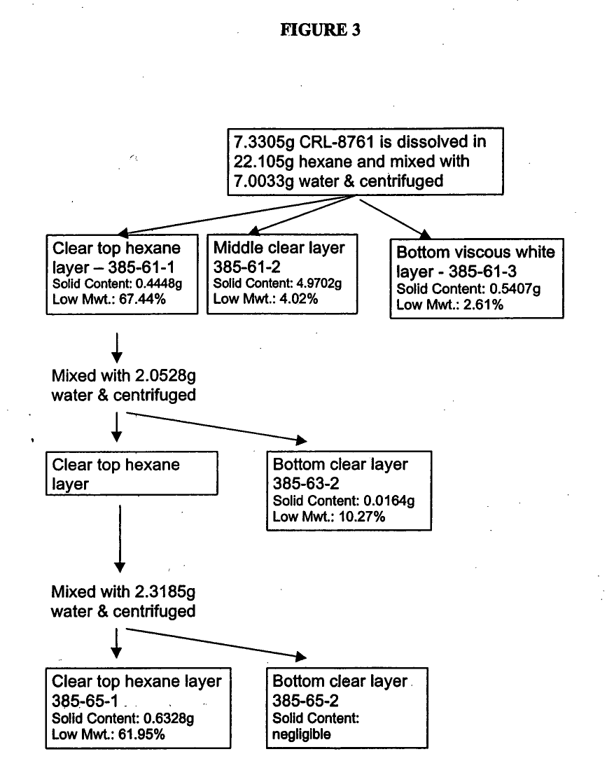

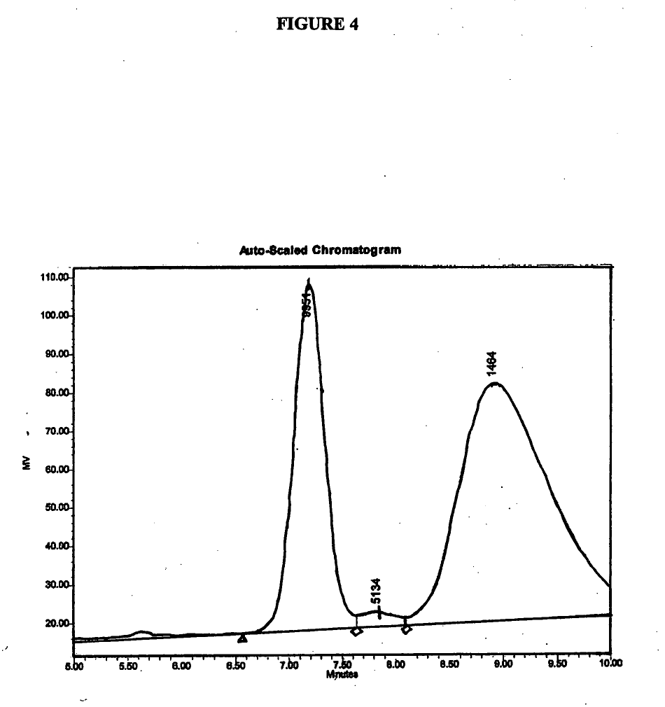

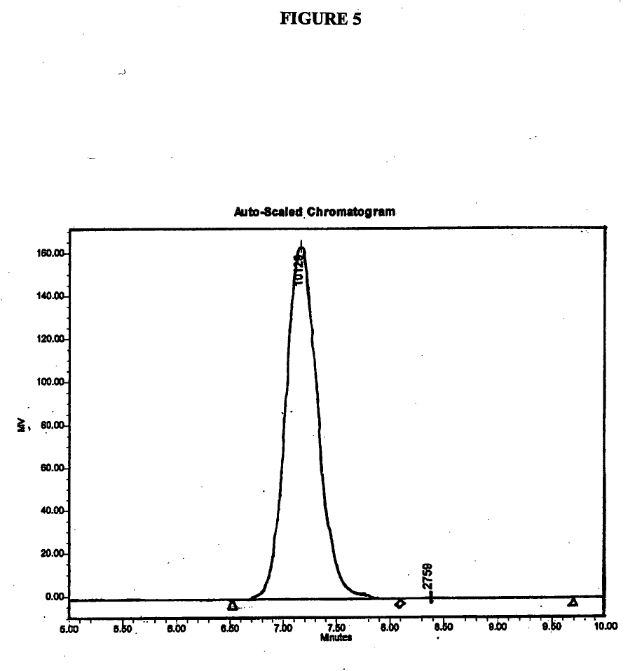

A second extraction was performed by extracting the top hexane layer with 2 g of water which was then centrifuged. Two layers were obtained. The bottom layer was sampled and dried to estimate the solid content. The top hexane layer from the previous wash was again extracted with 2.3 g of water then centrifuged. The two layers obtained were sampled and dried to estimate the solid content. This extraction sequence is depicted in FIG. 3. The results of the hexane extraction are shown below in Table 8. Chromatograms of a typical hexane layer (impurity enriched CRL-8761) and water layer (purified CRL-8761) are shown in FIGS. 4 and 5.

TABLE 8
Partition of CRL-8761 in first stage hexane extraction

| Starting | Hexane Layer from 1^{st} | Water Layers from 1^{st} |
|---|---|---|
| Material | Extraction | Extraction |
| CRL-8761 with | 6% of starting material | 75% of starting material |
| 6.74% low | with 67% low Mwt. | with 2.6-4.0% low |
| Mwt. content | component | Mwt. component |

*The solid contents in these fractions do not equal 100% due to experimental error and handling loss.

### Example 12

A simple solvent wash using three different hydrocarbon solvents was used to purify samples of CRL-8761. In a first wash, 1 g of CRL-8761 was placed in a 5 mL test tube and then mixed with 2 g of hexane. In a second wash, 1 g of CRL-8761 was placed in a 5 mL test tube and then mixed with 2 g of heptane. In a third wash, 1 g of CRL-8761 was placed in a 5 mL test tube and then mixed with 2 g of pentane. After vortex mixing, the copolymer/solvent mixtures were centrifuged. In each test tube, two layers were obtained. Each layer was sampled, dried, and analyzed by GPC. Two samples of the CRL-8761 used in each wash were also analyzed for comparison. The results are shown below in Table 9.

TABLE 9
Results of Simple Solvent Wash

| | | Peak | Low | % by |
|---|---|---|---|---|
| | | Molecular | Molecular | weight of |
| | | Weight | Weight | Low Molecular |
| | | Main Peak | Peak | Weight |
| Sample | Sample ID | (daltons) | (daltons) | Components |
| CRL-8761 | 385-68-8761 | 7332 | 1397 | 8.03 |
| CRL-8761 | 385-68-8761 | 7379 | 1438 | 6.7 |
| Hexane | 385-68-1XT | 7082 | 1347 | 43.02 |
| Top Layer | | | | |
| Hexane | 385-68-2XB | 7572 | <3063 | 5.0 |
| Bottom Layer | | | | |
| Heptane | 385-68-3HT | 6831 | 1344 | 63.24 |
| Top Layer | | | | |
| Heptane | 385-68-4HB | 7347 | <2966 | 5.66 |
| Bottom Layer | | | | |
| Pentane | 385-69-1PT | 6797 | 1327 | 49.85 |
| Top Layer | | | | |
| Pentane | 385-68-2PB | 7326 | <2919 | 6.19 |
| Bottom Layer | | | | |

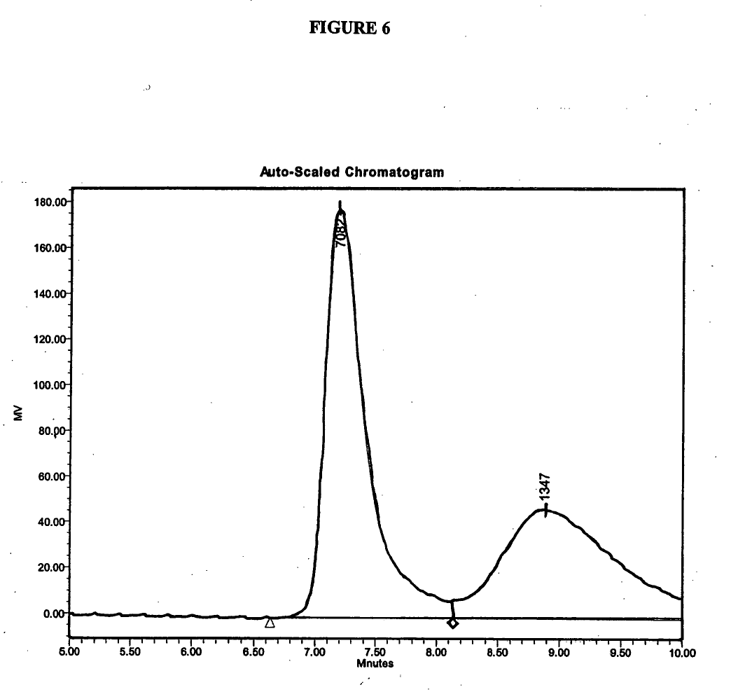

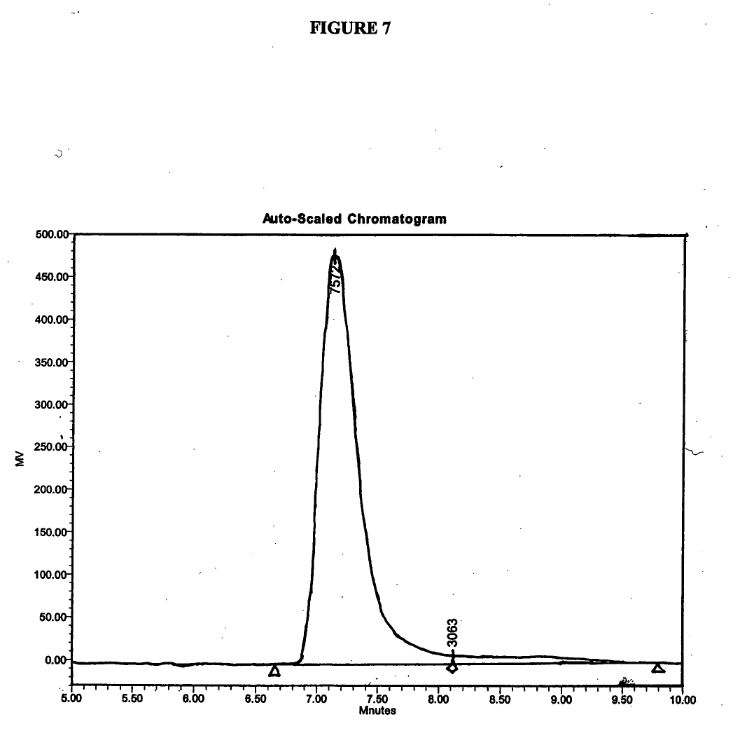

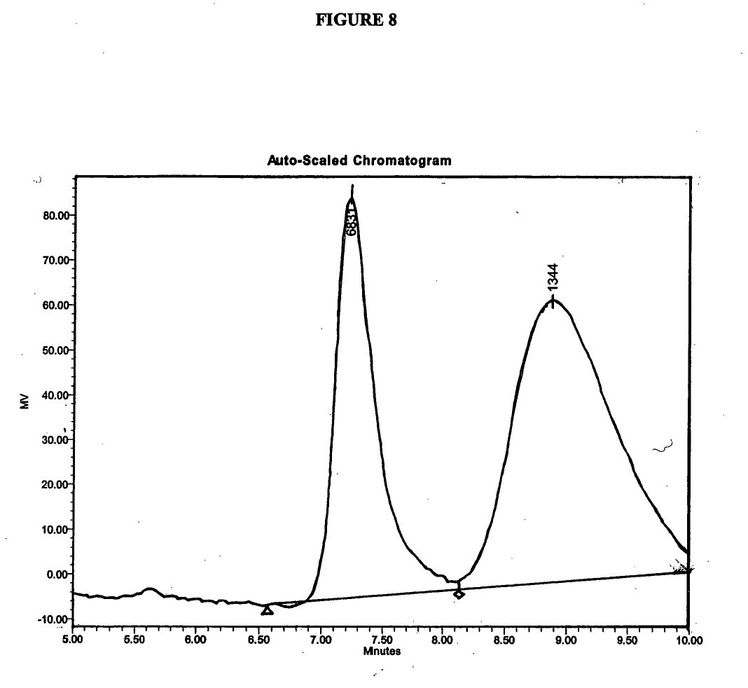

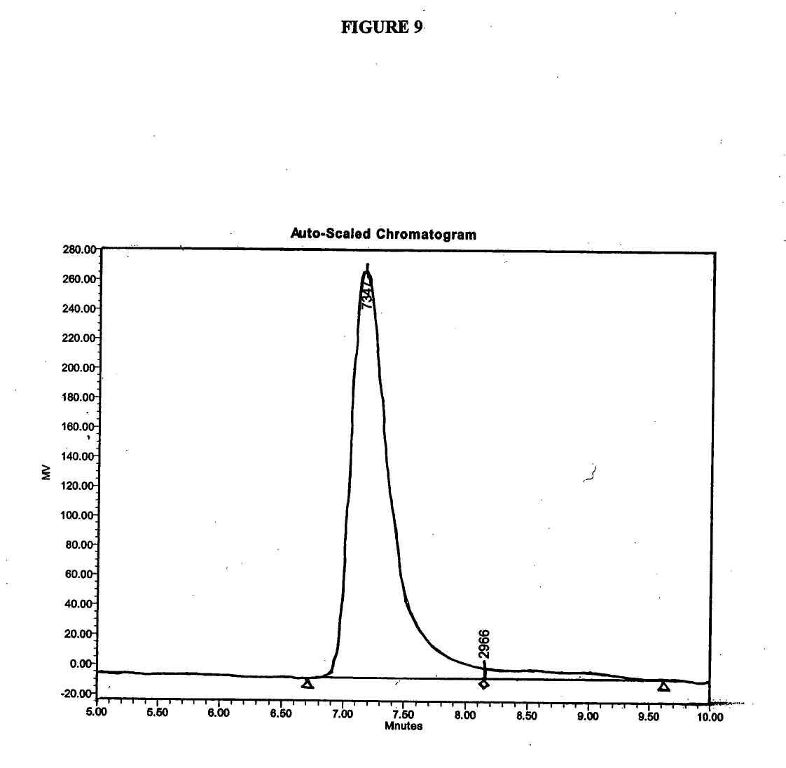

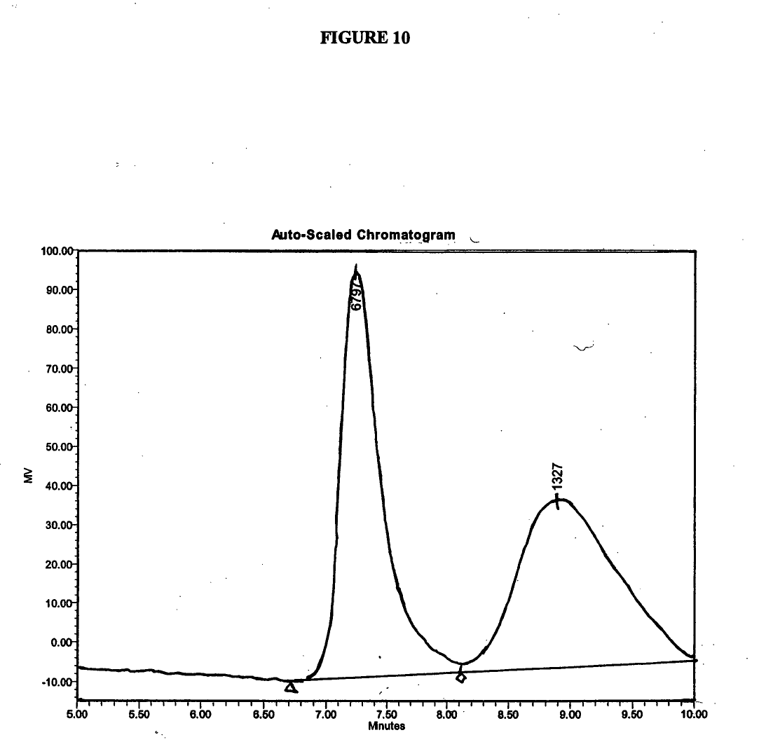

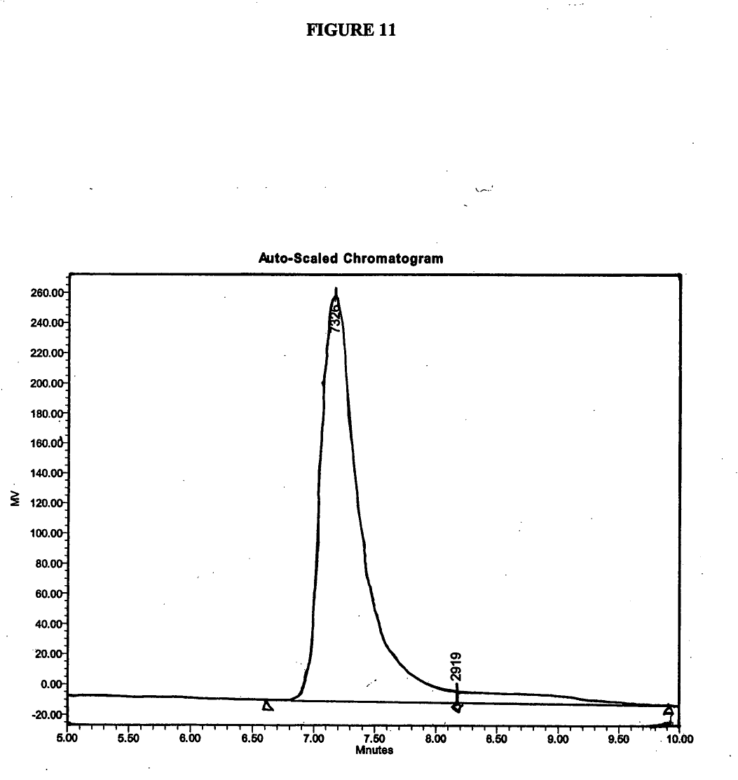

As shown in FIG. 6, the top hexane layer contained 10% of the CRL-8761 and showed a peak molecular weight of 7082 daltons wherein 43% of the layer contained low molecular weight components. As shown in FIG. 7, the bottom layer showed a peak molecular weight of 7572 daltons and only contained 5.0% by weight of low molecular weight components. As shown in FIG. 8, the top heptane layer had 6% of the CRL-8761 partitioned in. The peak molecular weight of the sample was 6831 daltons with 63% of the sample containing low molecular weight components. As shown in FIG. 9, the bottom layer showed a peak molecular weight of 7347 daltons and 5.7% low molecular weight components. As shown in FIG. 10, the top pentane layer had 10% of the CRL-8761 partitioned in. The peak molecular weight of the sample was 6797 daltons wherein 50% of the sample contained low molecular weight components. The bottom layer had a peak molecular weight of 7326 daltons and a percentage of low molecular weight components of 6.2% as shown in FIG. 11.

To further reduce the amount of low molecular weight components present in each sample, the purified copolymer from the bottom hexane, heptane and pentane layers is extracted and again washed with hexane, heptane and pentane, respectively. After separation, the samples are dried and analyzed by GPC to determine whether the amount of low molecular weight components present in the sample has been reduced below 4%, more preferably 3%, and most preferably 2%. The purified copolymer is repeatedly extracted and washed until the desired percentage of low molecular weight species is present in each sample.

### Example 13

In this example, a solvent wash using liquefied propane gas was used to purify samples of CRL-8761. Approximately 200 grams of CRL-8761 was loaded into a 1-L high-pressure stainless steel vessel equipped with a stirrer. While stirring the contents in the reactor, compressed propane was pumped into the reactor. The temperature of the propane fluid and the extraction vessel were maintained at 35° C. Initially, the propane pressure was maintained at 1,000 psia and approximately 100 Kg of SCF CO₂ were pumped over a period of 12 hours. The dissolved/extracted components were isolated from the solvent stream by lowering the propane pressure to approximately 400 psia to cause phase separation. The recovered propane was fed back to the solvent circulation loop. Approximately 2.2% of the CRL-8761 loaded into the reactor was removed by extraction. The extracted material contained approximately 75% low molecular weight components as measured by GPC. Following the extraction at 1,000 psia, the solvent pressure was raised to 1,500 psia and the extraction was continued for 10 more hours with 100 kg of CO₂ fluid pumped into the reactor over the 15 hour period. Approximately 5.5% of the CRL-8761 feed was removed by extraction. The extracted material contained approximately 60% low molecular weight components as measured by GPC. After the extraction was completed, the extraction vessel was depressurized and the purified CRL-8761 left in the vessel was analyzed by GPC. It contained approximately 1.6% of low molecular weight components. The yield of the purified CRL-8761 was approximately 82 wt. %.

### Example 14

In this example, a solvent wash using high pressure fluid carbon dioxide was used to purify samples of CRL-8761. Approximately 200 grams of CRL-8761 was loaded into a 1-L high-pressure stainless steel vessel equipped with a stirrer. While stirring the contents in the reactor, compressed fluid CO₂ was pumped into the reactor. The temperature of the CO₂ fluid and the extraction vessel were maintained at 35° C. Initially, the CO₂ pressure was maintained at 2,500 psia and approximately 100 Kg of SCF CO₂ were pumped over a period of 15 hours. The dissolved/extracted components were isolated from the solvent stream by lowering the CO₂ pressure to approximately 800 psia to cause phase separation. The recovered CO₂ was fed back to the solvent circulation loop. Approximately 2.4% of the CRL-8761 loaded into the reactor was removed by extraction. The extracted material contained approximately 85% low molecular weight components as measured by GPC. Following the extraction at 2,500 psia, the solvent pressure was raised to 3,500 psia and the extraction was continued for 15 more hours with 100 kg of CO₂ fluid pumped into the reactor over the 15 hour period. Approximately 4.5% of the CRL-8761 feed was removed by extraction. The extracted material contained approximately 55% low molecular weight components as measured by GPC. The extraction was further continued at 4,500 psia by pumping approximately additional 100 kg of CO₂ over a period of 15 hours. Approximately 10% of the CRL-8761 feed was removed. The extracted material contained approximately 25% low molecular weight components as measured by GPC. After the extraction was completed, the extraction vessel was depressurized and the purified CRL-8761 left in the vessel was analyzed by GPC. It contained approximately 2.4% of low molecular weight components. The yield of the purified CRL-8761 was approximately 78%.

### Example 15

In another example using high pressure fluid carbon dioxide as the extraction solvent, approximately 150 grams of CRL-8761 was mixed with 120 grams of Hydromatrix® diatomaceous earth (Varian, Inc., Palo Alto, Calif.) and then packed in a 500 ml high pressure extraction vessel. The vessel was connected to a high-pressure extraction system equipped with a solvent recycling capability. Approximately 100 kg of compressed fluid CO₂ was pumped into the extraction vessel over a period of 15 hours. The CO₂ extraction fluid and extraction vessel were maintained at a temperature of 35° C. and, initially, the CO₂ pressure was maintained at 2,500 psia. The dissolved/extracted components were isolated from the solvent stream by lowering the CO₂ pressure to approximately 800 psia to cause phase separation. The recovered CO₂ was then fed back to the solvent circulation loop. Approximately 2.1% of the CRL-8761 loaded into the vessel was removed by extraction. The extracted material contained approximately 88% low molecular weight components as measured by GPC. Following the extraction at 2,500 psia, the solvent pressure was raised to 3,500 psia and the extraction was continued for 15 more hours during which 100 kg of CO₂ fluid was pumped into the vessel. Approximately 4.1% of the initial charge was removed by this extraction method. The extracted material contained approximately 62% low molecular weight components as measured by GPC. The extraction was further continued at 4,500 psia by pumping an additional 100 kg of CO₂ into the vessel over a period of 15 hours. Approximately 12% of the initial charge into the reactor was removed by this method. The extracted material contained approximately 34% low molecular weight components as measured by GPC. After the extraction was completed, the extraction vessel was depressurized and the Hydromatrix/CRL-8761 mixture was washed with approximately 1 liter of ethanol. Purified CRL-8761 was isolated from the ethanol solution by evaporating the ethanol. The purified CRL-8761 was analyzed by GPC. It contained approximately 2.9% of low molecular weight components. The yield of the purified CRL-8761 was approximately 71%.

### Example 16

In this example, approximately 200 grams of CRL-8761 was loaded into a 1 L high pressure stainless steel vessel equipped with a stirrer. While stirring the contents in the reactor, compressed fluid CO₂ was pumped into the reactor. The temperature of the CO₂ fluid and the extraction vessel were maintained at 35° C. Compressed CO₂ mixed with 5-10% by weight methanol was used as the extraction solvent. Initially, the CO₂ pressure was maintained at 3,500 psia and approximately 60 kg of compressed CO₂/methanol extraction fluid mixture was pumped over a period of 10 hours. Methanol was pumped through a separate pump and mixed with compressed CO₂ prior to entering the extraction vessel. The flow rate for the methanol was adjusted to produce approximately 5% by weight of methanol in the CO₂ /methanol extraction fluid mixture. The dissolved/extracted components were isolated from the solvent stream by lowering the CO₂ pressure to approximately 800 psia to cause phase separation. The recovered CO₂ was fed back to the solvent circulation loop. Approximately 4% of the CRL-8761 loaded were removed by extraction. The extracted material contained approximately 76% low molecular weight components as measured by GPC. Following the extraction at 2,500 psia, the solvent pressure was maintained at 3,500 psia and the extraction was continued for 10 more hours with methanol concentration of 7% by weight in the extraction fluid and wherein 60 kg of CO₂/methanol fluid was pumped into the vessel. Approximately 7% by weight of the CRL-8761 charged were removed by extraction at this condition. The extracted material contained approximately 45% low molecular weight components as measured by GPC. The extraction was further continued at by pumping approximately additional 60 kg of CO₂ containing 9% by weight of methanol over a period of 10 hours. Approximately 13% of CRL-8761 charged into the reactor were removed under this condition. The extracted material contained approximately 24% low molecular weight components as measured by GPC. After the extraction was completed, the extraction vessel was depressurized. Methanol present in the mixture was evaporated and purified CRL-8761 was isolated and analyzed by GPC. It contained approximately 2.2% of low molecular weight components. The yield of the purified CRL-8761 was approximately 68%.

### Example 17

A purified polyoxypropylene/polyoxyethylene octablock copolymer was synthesized de novo by mixing approximately 25 g of Quadrol® (BASF Corporation, Mount Olive, N.J.)(ethylenediamine endcapped with 4 moles of ethylene oxide) and 1.25 g of potassium hydroxide in a glass liner placed inside a PARR reactor. The mixture was heated at 125° C. under vacuum for approximately three hours then the reactor temperature was reduced to approximately 90-100° C. and 105 g of ethylene oxide was slowly added over a period of 24 hours. After completing the ethylene oxide addition, approximately 600 g of propylene oxide was added using a metering pump. The internal pressure of the reactor was maintained at approximately between 20-30 psia. After the reaction was complete, approximately 12.5 g of Magnesol® (Dallas Group, White Hall, N.J.), 3.5 g of Celite® (World Minerals, Inc., Santa Barbara, Calif.), and 0.6 g of water were added to the reaction product over a period of six hours and in three different batches. The final product was allowed to cool to 40-50° C. and filtered through a pressure filter. The final product yield was approximately 600 grams with an estimated ethylene oxide content was approximately 15% by weight. The peak molecular weight of the final product was approximately 7100 daltons and the weight percent of low molecular weight components was approximately 1.23%.

### Example 18

In another example of the de novo synthesis of the purified composition of the present invention, 25 g of Quadrol® was mixed with 3.75 g of cesium hydroxide monohydrate in a glass liner placed inside a PARR reactor. The mixture was heated at 125° C. under vacuum for approximately six hours. The reactor temperature was reduced to approximately 90-100° C. and 105 g of ethylene oxide was slowly added over a period of 24 hours. After completing the ethylene oxide addition, approximately 600 g of propylene oxide was added using a metering pump. The internal pressure of the reactor was maintained at approximately between 20-30 psia. After the reaction was complete, approximately 25 g of Magnesol®, 7 g of Celite®g, and 1 g of water were added to the reaction product over a period of six hours and in three different batches. The final product was allowed to cool to 40-50° C. and filtered through a pressure filter. The final product yield was approximately 600 grams with an estimated percentage of ethylene oxide content in the product was approximately 15% by weight. The peak molecular weight of the product was approximately 7500 daltons and the weight percent of low molecular weight components in the product was approximately 1.13%.

### Example 19

A study is conducted at a commercial feed yard and utilizes 438 mixed-breed yearling steers with a mean initial body weight of 361 kg. Steers are obtained as a single group, sorted by body weight (BW) into two blocks of two pens each, and placed on feed. Within each pen, steers received either: (1) feed containing a sufficient amount of the purified copolymer of the present invention to inhibit growth of microorganisms and/or cause improved growth performance; or (2) feed containing a recommended dosage of conventional antibiotics and/or growth promotants. Steers are assigned to treatment on an every-other-head basis within each pen wherein the treatment assignment of the first steer in each pen is determined randomly. Cattle are weighed individually on the first day of the study. Pens are slaughtered 125 days (two heavy pens) or 141 days (two lighter pens) after the start of the study. Hot carcass weights (HCW) are collected immediately after evisceration. Individual animal average daily gains (ADG) are calculated using the equation: ADG=((HCW/0.635)−initial BW)/days on feed. In this equation, 0.635 represents the mean dressing percentage of all animals on the study. Twenty edible tissue samples are taken at random from each of the two groups of steers and tested for the presence of the copolymer of the present invention or the antibiotic and/or growth promotant. The test results demonstrate that the steers fed the copolymer of the present invention had comparable growth performance and food efficiency to the steers fed a traditional feed containing antibiotics and/or growth promotants and less than 50% of the copolymer was found in the edible tissue of the tested tissue samples.

1. A purified polyoxypropylene/polyoxyethylene block copolymer composition comprising the following formula:
(H(C₃H₆O)_{b}(C₂H₄O)ₐ)₂NC₂H₄N((C₂H₄O)ₐ(C₃H₆O)_{b}H)₂
wherein the molecular weight of the composition is from about 4000 to 10,000 daltons, “a” is a number such that the portion represented by polyoxyethylene constitutes from about 5-20% by weight of the composition, “b” is a number such that the portion represented by polyoxypropylene constitutes from about 80-95% by weight of the composition, and less than 4% by weight of the composition constitutes polymers having a molecular weight of less than 4000 daltons.

2. The composition of claim 1 wherein the molecular weight of the composition is from about 6000 to 9000 daltons.

3. The composition of claim 1 wherein the molecular weight of the composition is from about 7000 to 8000 daltons.

4. The composition of claim 1 wherein the polyoxyethylene portion constitutes from about 7-17% by weight of the composition.

5. The composition of claim 4 wherein the polyoxypropylene portion constitutes from about 83-93% by weight of the composition.

6. The composition of claim 1 wherein the polyoxyethylene portion constitutes from about 9-15% by weight of the composition.

7. The composition of claim 6 wherein the polyoxypropylene portion constitutes from about 85-91% by weight of the composition.

8. The composition of claim 1 wherein less than 2% by weight of the composition constitutes polymers having a molecular weight of less than 4000 daltons.

9. The composition of claim 1 wherein less than 1% by weight of the composition constitutes polymers having a molecular weight of less than 4000 daltons.

10. The composition of claim 1 wherein less than 4% by weight of the composition constitutes polymers having a molecular weight of less than 3000 daltons.

11. The composition of claim 1 wherein less than 4% by weight of the composition constitutes polymers having a molecular weight of less than 2000 daltons.

12. The composition of claim 8 wherein said polymers have a molecular weight of less than 3000 daltons.

13. The composition of claim 8 wherein said polymers have a molecular weight of less than 2000 daltons.

14. The composition of claim 9 wherein said polymers have a molecular weight of less than 3000 daltons.

15. The composition of claim 9 wherein said polymers have a molecular weight of less than 2000 daltons.

16. A method for preparing a purified polyoxypropylene/polyoxyethylene block copolymer composition comprising the steps of:
providing a polyoxypropylene/polyoxyethylene block copolymer composition having the following formula:
(H(C₃H₆O)_{b}(C₂H₄₀)ₐ)₂NC₂H₄N((C₂H₄O)ₐ(C₃H₆O)_{b}H)₂
wherein the mean molecular weight of the composition is from about 4000 to 10,000 daltons, “a” is a number such that the portion represented by polyoxyethylene constitutes from about 5-20% by weight of the composition, “b” is a number such that the portion represented by polyoxypropylene constitutes from about 80-95% by weight of the composition, and more than 4% by weight of the composition constitutes polymers having a molecular weight of less than 4000 daltons;
mixing the composition, water and a low-boiling, non-toxic solvent;
separating the mixture so that at least two layers are formed wherein at least one of the layers contains a purified polyoxypropylene/polyoxyethylene block copolymer composition having less than 4% by weight of polymers with a molecular weight of less than 4000 daltons; and
extracting the purified composition.

17. The method of claim 16 wherein the mean molecular weight of the composition is from about 6000 to 9000 daltons.

18. The method of claim 16 wherein the mean molecular weight of the composition is from about 7000 to 8000 daltons.

19. The method of claim 16 wherein the polyoxyethylene portion constitutes from about 7-17% by weight of the composition.

20. The method of claim 19 wherein the polyoxypropylene portion constitutes from about 83-93% by weight of the composition.

21. The method of claim 16 wherein the polyoxyethylene portion constitutes from about 9-15% by weight of the composition.

22. The method of claim 21 wherein the polyoxypropylene portion constitutes from about 85-91% by weight of the composition.

23. The method of claim 16 wherein less than 2% by weight of the purified composition constitutes polymers having a molecular weight of less than 4000 daltons.

24. The method of claim 16 wherein less than 1% by weight of the purified composition constitutes polymers having a molecular weight of less than 4000 daltons.

25. The method of claim 16 wherein less than 4% by weight of the composition constitutes polymers having a molecular weight of less than 3000 daltons.

26. The method of claim 16 wherein less than 4% by weight of the composition constitutes polymers having a molecular weight of less than 2000 daltons.

27. The method of claim 23 wherein said polymers have a molecular weight of less than 3000 daltons.

28. The method of claim 23 wherein said polymers have a molecular weight of less than 2000 daltons.

29. The method of claim 24 wherein said polymers have a molecular weight of less than 3000 daltons.

30. The method of claim 24 wherein said polymers have a molecular weight of less than 2000 daltons.

31. The method of claim 16 wherein the solvent is selected from the group consisting of high pressure or liquid carbon dioxide, acetone, alcohols, hydrocarbon solvents, and mixtures thereof.

32. The method of claim 31 wherein the hydrocarbon solvent is selected from the group consisting of methane, ethane, propane, butane, pentane, hexane, heptane, and mixtures thereof.

33. A method for preparing a purified polyoxypropylene/polyoxyethylene block copolymer composition comprising the steps of:
providing a polyoxypropylene/polyoxyethylene block copolymer composition having the following formula:
(H(C₃H₆O)_{b}(C₂H₄O)ₐ)₂NC₂H₄N((C₂H₄O)ₐ(C₃H₆O)_{b}H)₂
wherein the mean molecular weight of the composition is from about 4000 to 10,000 daltons, “a” is a number such that the portion represented by polyoxyethylene constitutes from about 5-20% by weight of the composition, “b” is a number such that the portion represented by polyoxypropylene constitutes from about 80-95% by weight of the composition, and more than 4% by weight of the composition constitutes polymers having a molecular weight of less than 4000 daltons;
mixing the composition and a low-boiling, non-toxic solvent;
separating the mixture so that at least two layers are formed wherein at least one of the layers contains a purified polyoxypropylene/polyoxyethylene block copolymer composition having less than 4% by weight of polymers with a molecular weight of less than 4000 daltons; and
extracting the purified composition.

34. The method of claim 33 wherein the molecular weight of the composition is from about 6000 to 9000 daltons.

35. The method of claim 33 wherein the molecular weight of the composition is from about 7000 to 8000 daltons.

36. The method of claim 33 wherein the polyoxyethylene portion constitutes from about 7-17% by weight of the composition.

37. The method of claim 36 wherein the polyoxypropylene portion constitutes from about 83-93% by weight of the composition.

38. The method of claim 33 wherein the polyoxyethylene portion constitutes from about 9-15% by weight of the composition.

39. The method of claim 38 wherein the polyoxypropylene portion constitutes from about 85-91% by weight of the composition.

40. The method of claim 33 wherein less than 2% by weight of the purified composition constitutes polymers having a molecular weight of less than 4000 daltons.

41. The method of claim 33 wherein less than 1% by weight of the purified composition constitutes polymers having a molecular weight of less than 4000 daltons.

42. The method of claim 33 wherein less than 4% by weight of the composition constitutes polymers having a molecular weight of less than 3000 daltons.

43. The method of claim 33 wherein less than 4% by weight of the composition constitutes polymers having a molecular weight of less than 2000 daltons.

44. The method of claim 40 wherein said polymers have a molecular weight of less than 3000 daltons.

45. The method of claim 40 wherein said polymers have a molecular weight of less than 2000 daltons.

46. The method of claim 41 wherein said polymers have a molecular weight of less than 3000 daltons.

47. The method of claim 41 wherein said polymers have a molecular weight of less than 2000 daltons.

48. The method of claim 33 wherein the solvent is selected from the group consisting of high pressure or liquid carbon dioxide, acetone, alcohols, hydrocarbon solvents, and mixtures thereof.

49. The method of claim 48 wherein the hydrocarbon solvent is selected from the group consisting of methane, ethane, propane, butane, pentane, hexane, heptane, and mixtures thereof.

50. A method for preparing a purified polyoxypropylene/polyoxyethylene block copolymer composition comprising the steps of:
providing a polyoxypropylene/polyoxyethylene block copolymer composition having the following formula:
(H(C₃H₆O)_{b}(C₂H₄O)ₐ)₂NC₂H₄N((C₂H₄O)ₐ(C₃H₆O)_{b}H)₂
wherein the mean molecular weight of the composition is from about 4000 to 10,000 daltons, “a” is a number such that the portion represented by polyoxyethylene constitutes from about 5-20% by weight of the composition, “b” is a number such that the portion represented by polyoxypropylene constitutes from about 80-95% by weight of the composition, and more than 4% by weight of the composition constitutes polymers having a molecular weight of less than 4000 daltons;
adding a quantity of high pressure carbon dioxide to said composition;
stirring the mixture under pressure;
separating the mixture to form at least two phases wherein a first phase contains said composition and a second phase contains a purified polyoxypropylene/polyoxyethylene block copolymer composition having less than 4% by weight of polymers with a molecular weight of less than 4000 daltons; and
extracting said first phase.

51. The method of claim 50 wherein the mean molecular weight of the composition is from about 6000 to 9000 daltons.

52. The method of claim 50 wherein the mean molecular weight of the composition is from about 7000 to 8000 daltons.

53. The method of claim 50 wherein the polyoxyethylene portion constitutes from about 7-17% by weight of the composition.

54. The method of claim 54 wherein the polyoxypropylene portion constitutes from about 83-92% by weight of the composition.

55. The method of claim 50 wherein the polyoxyethylene portion constitutes from about 9-15% by weight of the composition.

56. The method of claim 56 wherein the polyoxypropylene portion constitutes from about 85-91% by weight of the composition.

57. The method of claim 50 wherein less than 2% by weight of the purified composition constitutes polymers having a molecular weight of less than 4000 daltons.

58. The method of claim 50 wherein less than 1% by weight of the purified composition constitutes polymers having a molecular weight of less than 4000 daltons.

59. The method of claim 50 wherein less than 4% by weight of the composition constitutes polymers having a molecular weight of less than 3000 daltons.

60. The method of claim 50 wherein less than 4% by weight of the composition constitutes polymers having a molecular weight of less than 2000 daltons.

61. The method of claim 57 wherein said polymers have a molecular weight of less than 3000 daltons.

62. The method of claim 57 wherein said polymers have a molecular weight of less than 2000 daltons.

63. The method of claim 58 wherein said polymers have a molecular weight of less than 3000 daltons.

64. The method of claim 58 wherein said polymers have a molecular weight of less than 2000 daltons.

65. The method of claim 50 further comprising adding a quantity of a solvent to said carbon dioxide wherein said solvent is selected from the group consisting of acetone, alcohols, hydrocarbon solvents and mixtures thereof.

66. The method of claim 65 wherein the hydrocarbon solvent is selected from the group consisting of methane, ethane, propane, butane, pentane, hexane, heptane, and mixtures thereof.

67. The method of claim 50 further including the step of mixing the copolymer with a quantity of diatomaceous earth.

68. A method for preparing a purified polyoxypropylene/polyoxyethylene block copolymer composition comprising the steps of:
admixing respective quantities of a catalyst and a low molecular weight nitrogen compound;
adding a quantity of ethylene oxide;
adding a quantity of propylene oxide;
adding respective quantities of absorptive material to absorb catalyst, diatomaceous earth, and water;
heating the mixture; and
filtering the mixture through a pressure filter to produce a polyoxypropylene/polyoxyethylene block copolymer composition having the following formula:
(H(C₃H₆O)_{b}(C₂H₄O)ₐ)₂NC₂H₄N((C₂H₄O)ₐ(C₃H₆O)_{b}H)₂
wherein the mean molecular weight of the composition is from about 4000 to 10,000 daltons, “a” is a number such that the portion represented by polyoxyethylene constitutes from about 5-20% by weight of the composition, “b” is a number such that the portion represented by polyoxypropylene constitutes from about 80-95% by weight of the composition, and more than 4% by weight of the composition constitutes polymers having a molecular weight of less than 4000 daltons.

69. The method of claim 68 wherein the mean molecular weight of the composition is from about 6000 to 9000 daltons.

70. The method of claim 68 wherein the mean molecular weight of the composition is from about 7000 to 8000 daltons.

71. The method of claim 68 wherein the polyoxyethylene portion constitutes from about 7-17% by weight of the composition.

72. The method of claim 35 wherein the polyoxypropylene portion constitutes from about 83-92% by weight of the composition.

73. The method of claim 68 wherein the polyoxyethylene portion constitutes from about 9-15% by weight of the composition.

74. The method of claim 37 wherein the polyoxypropylene portion constitutes from about 85-91% by weight of the composition.

75. The method of claim 68 wherein less than 2% by weight of the purified composition constitutes polymers having a molecular weight of less than 4000 daltons.

76. The method of claim 68 wherein less than 1% by weight of the purified composition constitutes polymers having a molecular weight of less than 4000 daltons.

77. The method of claim 68 wherein less than 4% by weight of the composition constitutes polymers having a molecular weight of less than 3000 daltons.

78. The method of claim 68 wherein less than 4% by weight of the composition constitutes polymers having a molecular weight of less than 2000 daltons.

79. The method of claim 75 wherein said polymers have a molecular weight of less than 3000 daltons.

80. The method of claim 75 wherein said polymers have a molecular weight of less than 2000 daltons.

81. The method of claim 76 wherein said polymers have a molecular weight of less than 3000 daltons.

82. The method of claim 76 wherein said polymers have a molecular weight of less than 2000 daltons.

83. The method of claim 68 wherein the nitrogen compound is an amine alkoxylate.

84. The method of claim 83 wherein the amine alkoxylate is ethylenediamine.

85. A purified polyoxypropylene/polyoxyethylene block copolymer composition for administration to an animal comprising the following formula:
(H(C₃H₆O)_{b}(C₂H₄O)ₐ)₂NC₂H₄N((C₂H₄O)ₐ(C₃H₆O)_{b}H)₂
wherein the molecular weight of the composition is from about 4000 to 10,000 Daltons, “a” is a number such that the portion represented by polyoxyethylene constitutes from about 5-20% by weight of the composition, “b” is a number such that the portion represented by polyoxypropylene constitutes from about 80-95% by weight of the composition, and less than 50% by weight of the composition is absorbed through the gastrointestinal tract of the animal.

86. The composition of claim 85 wherein the molecular weight of the composition is from about 6000 to 9000 Daltons.

87. The composition of claim 85 wherein the molecular weight of the composition is from about 7000 to 8000 Daltons.

88. The composition of claim 85 wherein the polyoxyethylene portion constitutes from about 7-17% by weight of the composition.

89. The composition of claim 88 wherein the polyoxypropylene portion constitutes from about 83-92% by weight of the composition.

90. The composition of claim 85 wherein the polyoxyethylene portion constitutes from about 9-15% by weight of the composition.

91. The composition of claim 90 wherein the polyoxypropylene portion constitutes from about 85-91% by weight of the composition.

92. The composition of claim 85 wherein less than 4% by weight of the composition constitutes polymers having a molecular weight of less than 4000 Daltons.

93. The composition of claim 85 wherein less than 2% by weight of the composition constitutes polymers having a molecular weight of less than 4000 Daltons.

94. The composition of claim 85 wherein less than 1% by weight of the composition constitutes polymers having a molecular weight of less than 4000 Daltons.

95. The composition of claim 85 wherein less than 4% by weight of the composition constitutes polymers having a molecular weight of less than 3000 Daltons.

96. The composition of claim 1 wherein less than 4% by weight of the composition constitutes polymers having a molecular weight of less than 2000 Daltons.

97. The composition of claim 93 wherein said polymers have a molecular weight of less than 3000 Daltons.

98. The composition of claim 93 wherein said polymers have a molecular weight of less than 2000 Daltons.

99. The composition of claim 94 wherein said polymers have a molecular weight of less than 3000 Daltons.

100. The composition of claim 94 wherein said polymers have a molecular weight of less than 2000 Daltons.

101. The composition of claim 85 wherein less than 40% by weight of the composition is absorbed through the gastrointestinal tract of the animal.

102. The composition of claim 85 wherein less than 30% by weight of the composition is absorbed through the gastrointestinal tract of the animal.

103. The composition of claim 85 wherein less than 20% by weight of the composition is absorbed through the gastrointestinal tract of the animal.

104. The composition of claim 85 wherein less than 10% by weight of the composition is absorbed through the gastrointestinal tract of the animal.
